(19) United States
(12) Patent Application Publication
(10) Pub. No.: US 2006/0142744 A1
(43) Pub. Date: Jun. 29, 2006

(54) Identification connector for a medical laser handpiece

(76) Inventor: Dmitri Boutoussov, Dana Point, CA (US)

Correspondence Address: STOUT, UXA, BUYAN & MULLINS LLP, 4 VENTURE, SUITE 300, IRVINE, CA, 92618, US

(21) Appl. No.: 11/192,334

(22) Filed: Jul. 27, 2005

Related U.S. Application Data
(60) Provisional application No. 60/591,679, filed on Jul. 27, 2004.
(60) Provisional application No. 60/591,933, filed on Jul. 27, 2004.

(51) Int. Cl. A61B 18/18; A61C 1/00; A61C 3/00

(52) U.S. Cl. 606/10; 606/13; 606/15; 606/16; 433/29

(57) ABSTRACT

An identification connector is disclosed that provides a link between a laser housing and a laser handpiece. The connector integrates a laser delivery guide with ancillary connections and provides information for verifying proper connection and protection against use of unauthorized delivery systems.

### CROSS-REFERENCE TO RELATED APPLICATIONS

This application claims the benefit of U.S. Provisional Application No. 60/591,679, filed Jul. 27, 2004 and entitled IDENTIFICATION CONNECTOR, the entire contents of which are incorporated herein by reference. This application relates to U.S. Application No. 60/591,933, filed Jul. 27, 2004 and entitled CONTRA-ANGLE ROTATING HANDPIECE HAVING TACTILE FEEDBACK TIP FERRULE, the entire contents of which are incorporated herein by reference.

### BACKGROUND OF THE INVENTION

1. Field of the Invention

The present invention relates generally to electromagnetic energy emitting devices and, more particularly, to electromagnetic energy emitting devices that connect to various types of handpieces.

2. Description of Related Art

Laser devices used in dental and medical applications frequently employ handpieces that are specialized according to dental/medical applications. For example, a laser device and associated handpiece intended for use in liver surgery might vary significantly from another laser device intended for use in an orthopedic setting. In other cases, a basic laser housing containing electronics and hardware necessary to generate a laser beam may couple to a variety of handpieces that deliver laser energy to treatment sites. Ancillary functionality related to power settings, a need for illumination, a need to spray water or air, and other requirements may vary from one handpiece to another depending upon the specifics of a particular treatment plan.

Accidental use of a handpiece not properly matched to an intended medical or dental procedure could lead to undesirable consequences, including patient discomfort, damage to treated tissue, lost time, and the like.

A need exists in the prior art for methods and apparatuses that can provide a level of assurance that a laser handpiece is properly matched to an intended treatment device or technique. A further need exists for apparatuses that can provide various ancillary functionalities to support laser-based procedures in medical and dental applications.

### SUMMARY OF THE INVENTION

The present invention addresses these needs by providing an identification connector incorporated into a delivery system that can be connected to a laser housing. The laser housing may include an active identification device capable of receiving information from a passive identification device of the delivery system. The laser housing, which typically encloses a laser base unit, may further include a laser power coupling capable of providing laser power to the delivery system. A plurality of ancillary couplings also may be included in the laser housing. The identification connector may comprise, according to an illustrative embodiment of the present invention, the passive identification device capable of providing information to the active identification device. The identification connector further may comprise a laser beam delivery guide connection capable of receiving laser power from the laser power coupling. A typical embodiment of the identification connector further comprises a plurality of ancillary connections capable of connecting to the plurality of ancillary couplings.

The present invention further can comprise a method of connecting a delivery system to a laser housing. One implementation of the method can comprises providing an identification connector that includes a first identification device, the identification connector being connected to and forming a portion of the delivery system. The implementation further can comprise connecting the identification connector to the laser housing and receiving into the laser housing an indication according to the first identification device.

While the apparatus and method has or will be described for the sake of grammatical fluidity with functional explanations, it is to be expressly understood that the claims, unless expressly formulated under 35 U.S.C. 112, are not to be construed as necessarily limited in any way by the construction of “means” or “steps” limitations, but are to be accorded the full scope of the meaning and equivalents of the definition provided by the claims under the judicial doctrine of equivalents, and in the case where the claims are expressly formulated under 35 U.S.C. 112 are to be accorded full statutory equivalents under 35 U.S.C. 112.

Any feature or combination of features described herein are included within the scope of the present invention provided that the features included in any such combination are not mutually inconsistent as will be apparent from the context, this specification, and the knowledge of one skilled in the art. For purposes of summarizing the present invention, certain aspects, advantages and novel features of the present invention are described herein. Of course, it is to be understood that not necessarily all such aspects, advantages or features will be embodied in any particular embodiment of the present invention. Additional advantages and aspects of the present invention are apparent in the following detailed description and claims that follow.

### BRIEF DESCRIPTION OF THE FIGURES

FIG. 1 is a schematic diagram illustrating a link that connects a laser housing to a laser handpiece;

FIG. 2 is a simplified pictorial diagram depicting a delivery system comprising a laser handpiece and a link comprising an identification connector connected to a laser housing;

FIG. 3 is a pictorial diagram illustrating detail of an identification connector according to the present invention;

FIG. 4 is a perspective diagram of an embodiment of a housing module that may connect to a laser housing and that may accept an identification connector;

FIG. 5 is a front view of the embodiment of the housing module illustrated in FIG. 4;

FIG. 6 is a cross-sectional view of the housing module illustrated in FIG. 5, the cross-section being taken along a line 6-6′ of FIG. 5;

FIG. 7 is a flow diagram depicting an exemplary implementation of a method of connecting a laser housing to a delivery system according to the present invention;

FIG. 8 is a flow diagram illustrating an exemplary implementation of a method of providing a plurality of ancillary connections in an identification connector in accordance with the present invention;

FIG. 9 is a flow diagram describing an exemplary implementation of a method of providing a plurality of ancillary couplings in a laser housing; and

FIG. 10 is a flow diagram depicting an exemplary implementation of a method of receiving an indication from a first identification device.

### DETAILED DESCRIPTION OF THE PRESENT INVENTION

Reference will now be made in detail to the presently preferred embodiments of the invention, examples of which are illustrated in the accompanying drawings. Wherever possible, the same or similar reference numbers are used in the drawings and the description to refer to the same or like parts. It should be noted that the drawings are in simplified form and are not to precise scale. In reference to the disclosure herein, for purposes of convenience and clarity only, directional terms, such as, top, bottom, left, right, up, down, over, above, below, beneath, rear, and front, are used with respect to the accompanying drawings. Such directional terms should not be construed to limit the scope of the invention in any manner.

Although the disclosure herein refers to certain illustrated embodiments, it is to be understood that these embodiments are presented by way of example and not by way of limitation. The intent of the following detailed description, although discussing exemplary embodiments, is to be construed to cover all modifications, alternatives, and equivalents of the embodiments as may fall within the spirit and scope of the invention as defined by the appended claims. It is to be understood and appreciated that the process steps and structures described herein do not cover a complete process flow for operation of laser devices. The present invention may be practiced in conjunction with various techniques that are conventionally used in the art, and only so much of the commonly practiced process steps are included herein as are necessary to provide an understanding of the present invention. The present invention has applicability in the field of electromagnetic energy treatment devices in general. For illustrative purposes, however, the following description pertains to a medical laser device and a method of operating the medical laser device to perform tissue treatments and surgical functions.

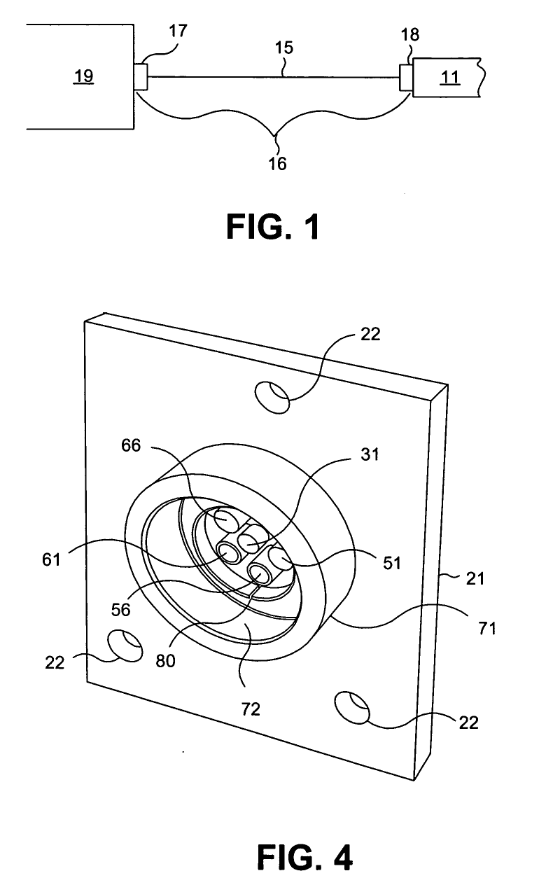

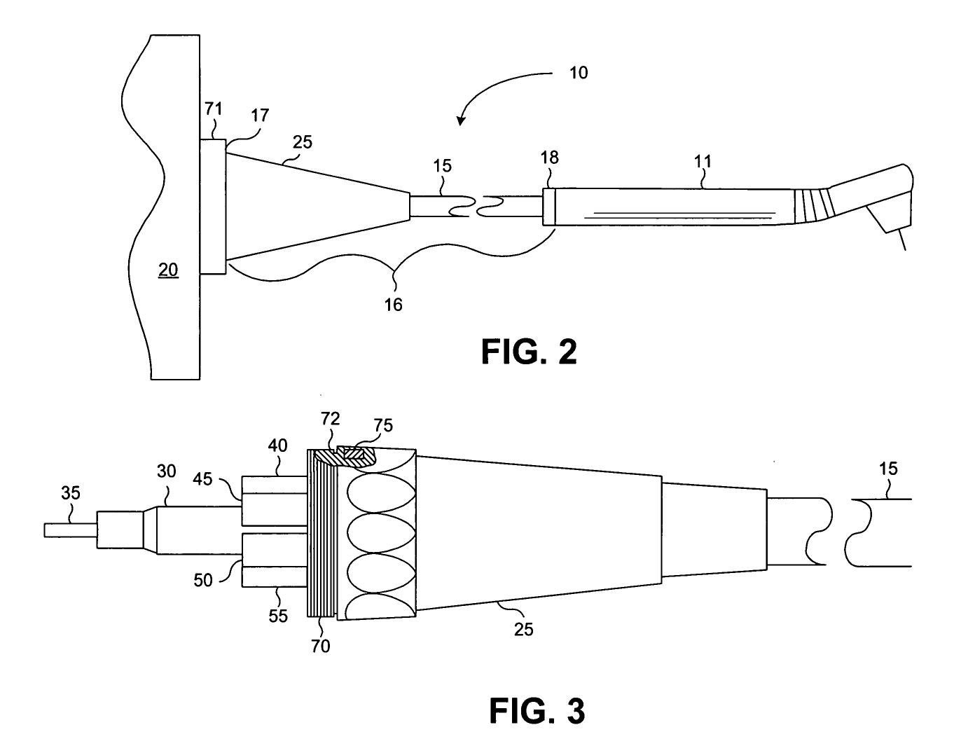

Referring more particularly to the drawings, FIG. 1 is a schematic diagram illustrating a link 16 that connects an energy source 19 to an output handpiece 11 according to the present invention. The link 16 comprises a first end 17 that connects to the energy source 19, a conduit 15, and a second end 18 that connects to the output handpiece 11. An exemplary embodiment of a link 16 is further illustrated and described in connection with FIG. 2.

FIG. 2 is a simplified pictorial diagram depicting an exemplary embodiment of the above-described system of FIG. 1, wherein corresponding or like elements are labeled with like reference number designators. A delivery system 10 comprises an electromagnetic energy (e.g., laser) handpiece 11 and a link 16 including an identification connector 25 connected to a laser housing 20 at a first end 17 in accordance with the present embodiment. The link 16 further comprises a conduit 15 that provides coupling between the identification connector 25 and the laser handpiece 11 at a second end 18. According to an exemplary implementation, the conduit 15 comprises channels (not shown) capable of carrying laser power, fluid (e.g., spray air and spray water), illumination, and the like to the laser handpiece 11. The conduit 15 further may comprise a feedback channel capable of carrying feedback information (e.g., optical feedback) from the laser handpiece 11 to the identification connector 25. The identification connector 25 may connect mechanically to the laser housing 20 with, for example, a threaded connection (cf. 70, FIG. 3) to a receptacle 71 that forms part of the laser housing 20.

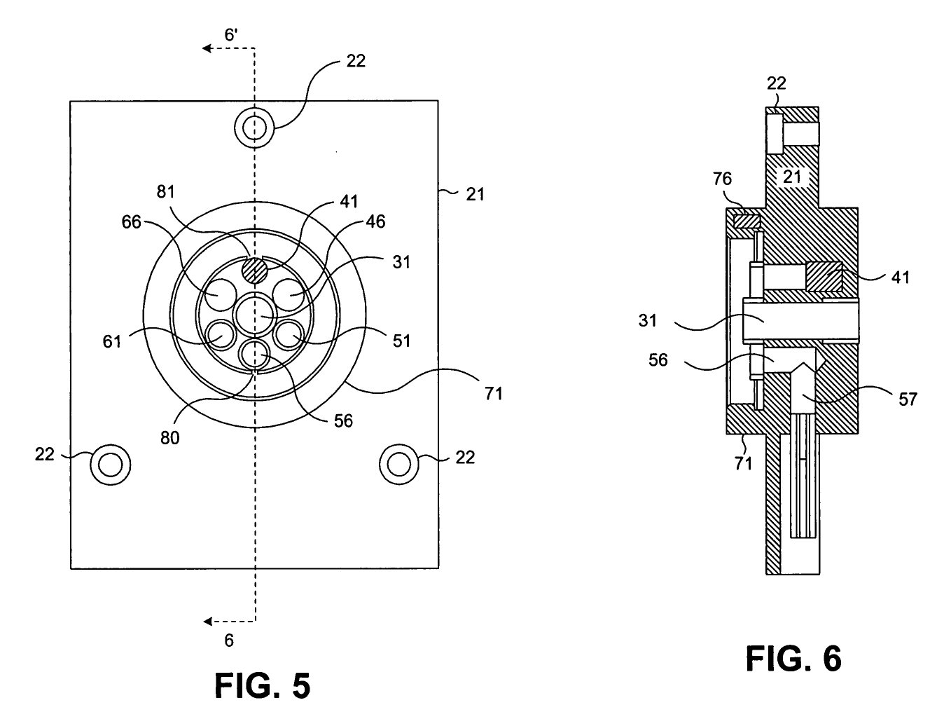

An exemplary implementation of an identification connector 25 is further illustrated in FIG. 3. The illustrated embodiment comprises an electromagnetic energy beam delivery guide connection, which, in a typical embodiment may comprise a laser beam delivery guide connection 30 including, for example, an optical fiber 35 capable of transmitting a laser beam to the laser handpiece 11 (FIG. 2). The illustrated embodiment further comprises a plurality of ancillary connections comprising, in this example, a feedback connection 40, an illumination light connection 45, a spray air connection 50, and a spray water connection 55, that may connect to the laser housing 20 (FIG. 2) enclosing an electromagnetic energy (e.g., laser) base unit (not shown). The plurality of ancillary connections further may comprise connections not visible in FIG. 3 such as a cooling air connection (accommodated to connect with cooling air coupling 61, FIG. 5) and an excitation light connection (accommodated to connect with excitation light coupling 66, FIG. 5).

The embodiment of the identification connector illustrated in FIG. 3 comprises a threaded portion 70 that may mate with and thereby provide for connection to the receptacle 71 (FIG. 2) on the laser housing 20. The embodiment still further comprises an information communication element, such as an identification device or, as presently embodied, a passive identification device 75 shown embedded in the identification connector 25 in a cut-away portion 72 of the diagram. The passive identification device 75 may store in, for example, nonvolatile memory such as flash memory, information such as identification information pertaining to a type of identification connector 25 and a type of link 16 (FIGS. 1 and 2) that comprises the identification connector 25. The passive identification device 75 further may store operation information such as a calibration factor that may be used, for example, to adjust laser power directed through the laser beam delivery guide connection 30. In other embodiments, the identification device can comprise an active identification device including, for example, a microprocessor and power supply.

FIG. 4 is a perspective diagram of an embodiment of a housing module that may connect to a laser housing 20 (FIG. 2) and that further may accept an identification connector 25. The illustrated embodiment comprises a plate 21 that may fasten to a laser housing 20 by means of, for example, screws inserted into apertures 22. The housing module comprises a receptacle 71 that may be threaded on an inside surface 72 to mate with threads 70 (FIG. 3) of the identification connector 25. The embodiment of the housing module further comprises a laser power coupling 31 that is constructed to accommodate the laser beam delivery guide connection 30 (FIG. 3), the laser power coupling 31 being capable of providing laser power to the delivery system 10. The embodiment further comprises a plurality of ancillary couplings including spray air coupling 51, a spray water coupling 56, a cooling air coupling 61, and an excitation light coupling 66. The embodiment still further comprises a feedback detector (cf. 41, FIG. 5) and an illumination light coupling (cf. 46, FIG. 5) that are not visible in the diagram. One or more alignment provisions or structures, such as key slots 80 and 81 provided in the illustrated embodiment, may be included to assure that the identification connector 25 is connected to the receptacle 71 in a correct orientation.

FIG. 5 is a front view of the embodiment of the housing module illustrated in FIG. 4. The view in FIG. 5 illustrates the plate 21 and the apertures 22 that may be used to secure the housing module to a laser housing. Further illustrated are the laser power coupling 31, feedback detector 41, illumination light coupling 46, the spray air coupling 51, the spray water coupling 56, the cooling air coupling 61, and the excitation light coupling 66. In operation, the spray water coupling 56 mates with and is capable of supplying spray water to the spray water connection 55 in the identification connector 25 (FIG. 3). Similarly, the spray air coupling 51 mates with and is capable of supplying spray air to the spray air connection 50 in the identification connector 25. Additionally, the illumination light coupling 46, the excitation light coupling 66, and the cooling air coupling 61 mate with and are capable of supplying, respectively, illumination light to the illumination light connection 45, excitation light to the excitation light connection (not shown), and cooling air to the cooling air connection (not shown) in the identification connector 25. Further, the feedback detector 41 mates with and is capable of receiving feedback from the feedback connection 40 in the identification connector 25. According to an illustrative embodiment, the illumination light coupling 46 and the excitation light coupling 66 couple light from, for example, a light-emitting diode (LED) or a laser light source to the respective illumination light connection 46 and the excitation light connection (not shown). One embodiment employs two white LEDs as a source for illumination light. As described above with reference to FIG. 4, according to the illustrated embodiment one or more key slots 80 and 81 may be provided to prevent the identification connector 25 from being connected to the receptacle 71 in an incorrect or inoperative orientation.

FIG. 6 is a cross-sectional view of the housing module illustrated in FIGS. 4 and 5. The cross-section is taken along a line 6-6′ in FIG. 5, the line 6-6′ showing cross-sections of the feedback detector 41, the laser power coupling 31, and the spray water coupling 56. A water source 57 may supply water to the water coupling 56.

Additional sources for ancillary functions further may be provided in the housing module illustrated in FIGS. 4-6. For example, one or more combining or selecting elements (not shown) may be provided to enable switching between, for example, electromagnetic energy outputs of varying characteristics. Such electromagnetic energy outputs of varying characteristics can be provided to one or more of the illumination light coupling 46 and the excitation light coupling 66 in varying degrees (e.g., varying intensities) and/or at varying times (e.g., with electromagnetic outputs being switched to or between the illumination light coupling 46 and the excitation light coupling 66 at times and with appropriate characteristics according to times when the illumination light coupling 46 and the excitation light coupling 66 are used and according to various types of uses).

In typical embodiments, the combining or selecting elements can be disposed between electromagnetic energy sources and one or more of the illumination light connection 45 and the excitation light connection (not shown). In other embodiments, the combining or selecting elements can be disposed between electromagnetic energy sources and one or more of the illumination light coupling 46 and the excitation light coupling 66. The electromagnetic energy outputs may vary spatially and/or temporally in wavelength, wavelength distribution, intensity, intensity distribution, and combinations thereof. An exemplary embodiment of the combining or selecting elements can comprise a plurality of pneumatic shutter filters, positioned between electromagnetic energy sources and one or more of the illumination light coupling 46 and the excitation light coupling 66, and being configured to facilitate switching between blue and white light that is coupled to one or more of the illumination light coupling 46 and the excitation light coupling 66 in order to alter or enhance excitation and visualization functions. According to another embodiment, the pneumatic shutters may switch between varying amounts or other characteristic(s) of light, and/or may switch between varying sources of light, such as switching between white light and any other (filtered) color of light.

The cross-sectional view of FIG. 6 further illustrates an information communication element, such as an identification device or, as presently embodied, an active identification device 76 embedded in the housing module. In various embodiments, either or both of the information communication element of the identification connector 25 (FIG. 3) and the information communication element of the housing module (FIG. 6) may be implemented or eliminated. In an illustrative embodiment, information communication elements disposed in both the identification connector and the housing module are implemented as a passive identification device 75 (FIG. 3) and an active identification device 76 (FIG. 6), respectively. The active identification device 76 may interrogate the passive identification device 75 when, for example, the identification connector 25 is connected to the laser housing 20 (FIG. 2).

Communications or interactions to and from one or more of the information communication element of the identification connector 25 (FIG. 3) and the information communication element of the housing module (FIG. 6) may comprise the transmission or receipt of operation power or information using structure and protocols known to those skilled in the art. In an exemplary embodiment wherein the information communication element of the identification connector 25 comprises a passive identification device 75 and the information communication element of the housing module comprises an active identification device 76, items such as operation power for enabling operation of the passive identification device 75 and/or information such as identification information from the passive identification device 75 can be supplied (e.g., via conductors or inductive coupling) from the active identification device 76 to the passive identification device 75 when the identification connector 25 of the delivery system 11 (FIG. 2) is connected to the receptacle 71 of the laser housing 20. The passive identification device 75 may have stored therein (e.g., in flash memory or in read-only memory coded at time of manufacture), a serial number associated with the identification connector 25. The passive identification device 75 further may have stored therein a calibration factor that may be employed by the laser base unit within the laser housing 20 (FIG. 2) to adjust, for example, laser power. In other implementations, parameters of or within the laser housing 20 (e.g., a pulse rate, a type of display or a type or format of information provided to a user) may be provided based upon information received from the passive identification device 75. The parameters may comprise a type or characteristic of one or more of the delivery system and the identification connector. The parameters may be received, in whole or in part, from one or more of the delivery system and the identification connector 25. Further, one or more of the passive identification device 75 and the active identification device 76 may include a counter, such as a pulse counter, that may accumulate data, such as use data (e.g., a number of pulses delivered by a delivery system or component (e.g., a fiber optic) thereof. Based at least in part upon parameters (e.g., a number of pulses) received from or communicated by one or more of the passive identification device 75 and the active identification device 76.

In an illustrative embodiment, the passive identification device 75 accumulates a number of pulses that have been delivered by a particular fiber optic that is attached to the delivery system 10. When the identification connector 25 is coupled to the delivery system 11, the passive identification device 75 conveys this count information to the active identification device 76, which then monitors a number of pulses that are provided from the laser base unit within the laser housing 20 to the delivery system 11 while that same fiber optic remains connected. The laser housing 20 can initiate preventive maintenance by, for example, activating a service warning indicator according to a number of laser pulses that have been generated while the identification connector 25 was connected to the fiber optic being monitored. If the fiber optic being monitored is removed from the delivery system 10 and replaced with another fiber optic, count information being accumulated within the laser base unit can be modified (e.g., reset) according to relevant count information of the new fiber optic. When the identification connector 25 is disconnected from the laser housing 20, or at other predetermined or event-triggered times, a count accumulated by the active identification device 76 can be communicated to and stored on the passive identification device 75. According to other aspects of the invention, when a serial number is stored in the passive identification device 75, the serial number may result in the laser base unit preventing access to or use of one or more functions of the laser base unit or the delivery system when, for example, an unauthorized delivery system is connected to the laser housing. For example, connecting to the laser base unit an identification connector having mechanical characteristics similar to those of the identification connector 25 but without a passive identification device 75 may cause the laser base unit to shut down laser-beam functionality. The shut-down may occur for safety and other business reasons. The laser base unit may behave similarly when an identification connector 25 having a passive identification device 75 is connected when the serial number of the passive identification device 75 is not authorized.

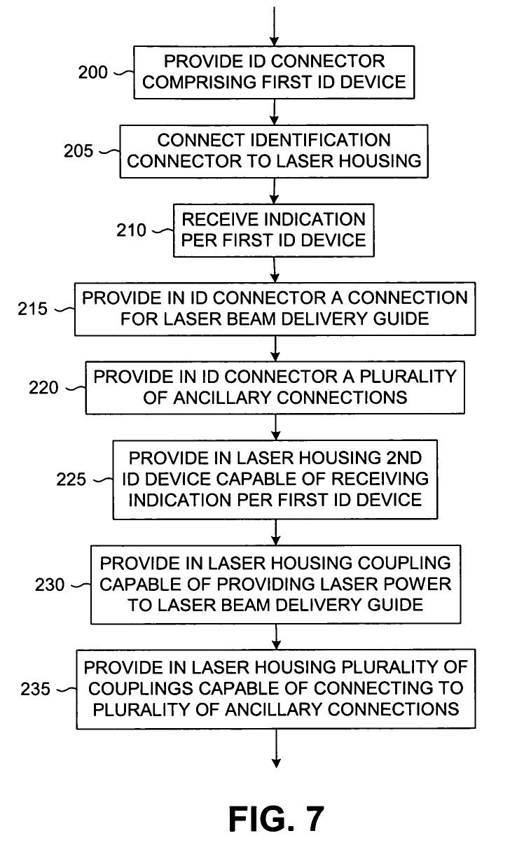

One aspect of the present invention comprises a method of connecting a laser housing to a delivery system. FIG. 7 is a flow diagram depicting an implementation of the method, wherein, for example, a housing module of a laser housing 20 (FIG. 2) can be connected to an identification connector of a delivery system. The illustrated implementation comprises providing an identification connector at step 200, which step can be implemented in an order different from that depicted in FIG. 7, wherein the identification connector comprises a first identification device that in certain implementations may comprise a passive identification device. An exemplary embodiment of an identification connector 25 is illustrated in FIGS. 2 and 3. Further, the identification connector 25 may comprise a first identification device, such as the passive identification device 75 illustrated in FIG. 3. The identification connector is connected to a laser housing at step 205 of the implementation described in FIG. 7. For example, the identification connector may be connected to a laser housing 20 as shown in FIG. 2 wherein the laser housing 20 encloses a laser base unit.

Upon connection of the identification connector to the laser housing, an indication may be received according to the first identification device at step 210. The illustrated implementation continues by providing in the identification connector a connection for a laser beam delivery guide at step 215, which step can be implemented in an order different than that depicted in FIG. 7. The laser beam delivery guide connection 30 illustrated in FIG. 3 illustrates one example of such a connection. A plurality of ancillary connections further is provided in the identification connector at step 220, which step can be implemented in an order different from that depicted in FIG. 7. As an example, the embodiment of the identification connector shown in FIG. 3 includes a plurality of ancillary connections including, for example, a feedback connection 40, an illumination light connection 45, a spray air connection 50 and a spray water connection 55.

The illustrated implementation of the method of the present invention continues by providing elements in the laser housing that correspond with elements of the identification connector. In particular, at step 225, which step can be implemented in an order different from that depicted in FIG. 7, there is provided in the laser housing a second identification device, which may comprise an active identification device, capable of receiving an indication according to the first identification device. The second identification device may be implemented, for example, as depicted in FIG. 6 wherein an active identification device 76, which may be capable, according to an exemplary embodiment, of receiving an indication from a first identification device, is illustrated. For example, the active identification device 76 may be capable of receiving an indication from a first identification device such as the passive identification device 75 illustrated in FIG. 3.

A coupling capable of providing laser power to a laser beam delivery guide connection in an identification connector may be provided in the laser housing at step 230, which step can be implemented in an order different than that depicted in FIG. 7. An example of such a coupling is described above in the context of a laser power coupling 31 with reference to FIGS. 4-6. In the illustrated implementation of the method, a plurality of couplings capable of connecting to the plurality of ancillary connections may be provided at step 235, which step can be implemented in a different order than that depicted in FIG. 7. Such couplings may include, for example, as illustrated in FIG. 5, a feedback detector 41 capable of receiving feedback from a feedback connection 40 (FIG. 3), an illumination light coupling 46 capable of supplying illumination light to an illumination light connection 45, a spray air coupling 51 capable of supplying spray air to a spray air connection 50, a spray water coupling 56 capable of supplying spray water to a spray water connection 55, a cooling air coupling 61 capable of supplying cooling air to a cooling air connection (not shown), and an excitation light coupling 66 capable of supplying excitation light to an excitation light connection (not shown).

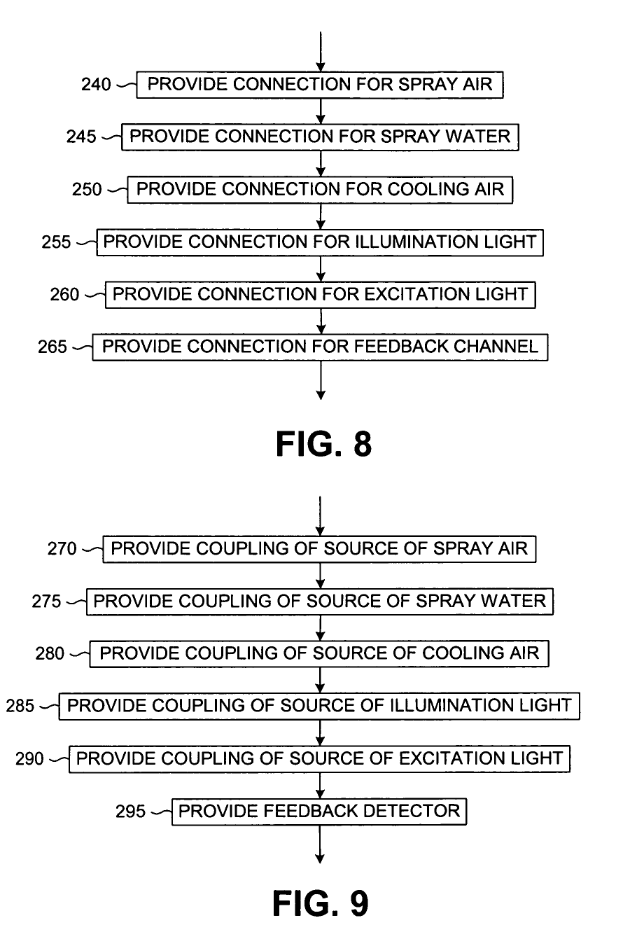

FIG. 8 is a flow diagram depicting steps, which may vary in number, combination or order in modified embodiments, of an exemplary implementation of a method of providing a plurality of ancillary connections (cf. step 220 in FIG. 7) according to the present invention. A connection for spray air is provided at step 240. Likewise, a connection for spray water is provided at step 245. Additional connections for, respectively, cooling air, illumination light, and excitation light are provided at steps 250, 255, and 260. A connection for a feedback channel is provided at step 265.

As suggested above with reference to step 235 of the implementation of the method described in FIG. 7, a plurality of couplings may be provided, not necessarily in any particular number, combination or order, in a laser housing, wherein the couplings are capable of connecting to the connections provided in the steps of the implementation described in FIG. 8. As illustrated in a flow diagram in FIG. 9, a coupling of a source of spray air may be provided at step 270, the spray air coupling being capable of connecting to the connection for spray air provided at step 240 above. A coupling of a source of spray water also may be provided at step 275, wherein the spray water coupling is capable of connecting to the connection for spray water provided at step 245. Likewise, a coupling of a source of cooling air may be provided at step 280. The coupling may be capable of connecting to the connection for cooling air provided at step 250. Similarly, couplings for sources of, respectively, illumination light and excitation light may be provided at steps 285 and 290, these couplings being capable of connecting to the connections for illumination light and excitation light at steps 255 and 260. Further, a feedback detector may be provided at step 295. The feedback detector may be capable of receiving feedback from the connection for a feedback channel provided in step 265. The respective connections provided in the steps illustrated in FIG. 8 may take a form as illustrated in FIG. 3 wherein connections for a feedback channel 40, illumination light 45, spray air 50, and spray water 55 are illustrated. Connections for cooling air and excitation light, although included in the illustrated embodiment, are not shown explicitly in FIG. 3. Similarly, the couplings provided in the steps of the implementation depicted in FIG. 9 may be the same as or similar to the couplings illustrated in FIG. 5. The housing module shown in FIG. 5 includes a feedback detector 41 and couplings for illumination light 46, spray air 51, spray water 56, cooling air 61 and excitation light 66.

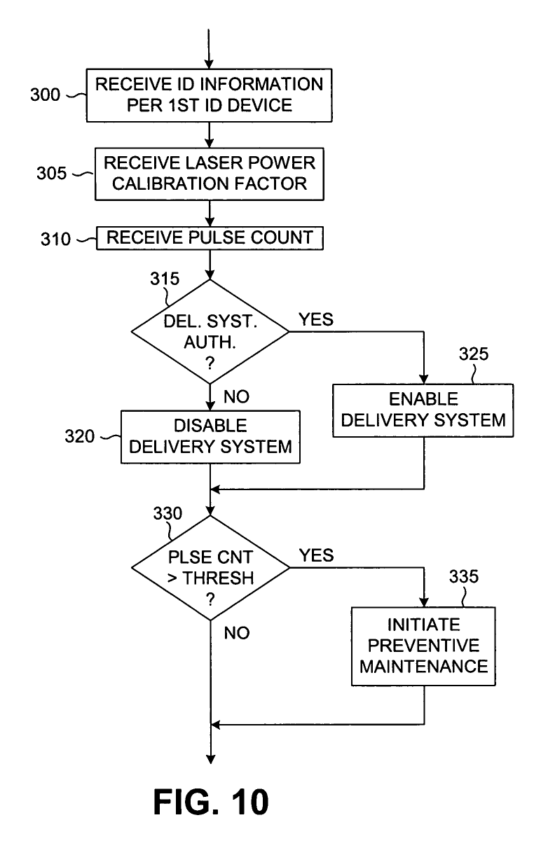

FIG. 10 is a flow diagram depicting steps, which may vary in number, combination or order in modified embodiments, of an implementation of a method of receiving an indication from the first identification device (cf. step 210 in FIG. 7). The illustrated implementation comprises receiving identification information according to the first identification device at step 300. For example, identification information comprising a serial number may be received from the passive identification device 75 illustrated in FIG. 3 when the identification connector 25 is connected to the laser housing 20 (FIG. 2). A laser power calibration factor further may be received at step 305. In an exemplary embodiment, the laser power calibration factor is associated with the identification connector 25 (FIGS. 2 and 3) and may enable a laser base unit to adjust a power level according to a connected identification connector 25 and associated laser handpiece 11. A pulse count may be received, according to an illustrative implementation, by the first identification device at step 310. In one embodiment, the pulse count can comprise a cumulative number of pulses of laser energy (a) transmitted through the identification connector (of a delivery system) and/or (b) transmitted through a given fiber optic while that fiber optic has been coupled to the delivery system. In one embodiment, the pulse count (or information pertaining thereto) is received into the second identification device, and subsequently updated according to a cumulative number of pulses of laser energy produced by the laser base unit while connected to the identification connector, and subsequently provided back to the first identification device. In another embodiment, the pulse count is received into the second identification device, and subsequently updated according to a cumulative number of pulses of laser energy produced by the laser base unit while connected to the identification connector with the same fiber optic connected thereto, and subsequently provided back to the first identification device. Consequently, by storing a pulse count for each of, perhaps, several identification connectors (and/or fiber optics connected thereto), separable according to their serial numbers, the second identification device may accumulate usage information regarding preventive maintenance with respect to each identification connector. For example, when a number of pulses exceeds a predetermined threshold, a warning may be displayed on the laser housing indicating that one of an identification connector, a fiber optic, and an associated laser handpiece should be inspected, discarded, or the like.

The implementation shown in FIG. 10 continues by testing whether a delivery system is authorized at step 315. For example, the second identification device may, upon receiving a serial number from the first identification device, determine that the identification connector is not authorized for use with the laser housing and laser base unit. Such nonauthorization may occur, for example, when a “Brand-X” identification connector is substituted for an authorized identification connector compatible with the laser base unit. According to another example, a laser base unit may support a plurality of types of delivery systems according to a type of procedure to be performed, such as cutting of hard or soft tissue, reforming of dental tissue, and the like. For example, hard tissue may include enamel, dentin, and bone whereas soft tissue may comprise, for example, periodontal, mucosa, dermal tissue, liver tissue, and cardiac tissue. Each of these types of procedures may require a different type of delivery system, and the present invention may help to assure that the appropriate delivery system is used for a given procedure. When the delivery system is authorized at step 315, the delivery system may be enabled at step 325. For example, laser power may be provided to the delivery system according to the calibration factor received at step 305. When the delivery system is not authorized at step 315, the delivery system may be disabled at step 320. One way of disabling the delivery system is to inhibit the delivery of laser power to the delivery system. The pulse count received at step 310 of the implementation may be compared with a predetermined threshold at step 330. If the pulse count exceeds the predetermined threshold, then preventive maintenance may be initiated at step 335. Initiation of preventive maintenance may include, for example, displaying a warning on a user console indicating that preventive maintenance should be performed.

The identification connector of the present invention can be used, for example, with U.S. Application No. 60/591,933, filed Jul. 27, 2004 and entitled CONTRA-ANGLE ROTATING HANDPIECE HAVING TACTILE FEEDBACK TIP FERRULE. Corresponding or related structure and methods described in the following patents assigned to BioLase Technology, Inc. are incorporated herein by reference in their entireties, wherein such incorporation includes corresponding or related structure (and modifications thereof) in the following patents which may be (i) operable with, (ii) modified by one skilled in the art to be operable with, and/or (iii) implemented/used with or in combination with any part(s) of, the present invention according to this disclosure, that/those of the patents, and the knowledge and judgment of one skilled in the art: U.S. Pat. No. 5,741,247; U.S. Pat. No. 5,785,521; U.S. Pat. No. 5,968,037; U.S. Pat. No. 6,086,367; U.S. Pat. No. 6,231,567; U.S. Pat. No. 6,254,597; U.S. Pat. No. 6,288,499; U.S. Pat. No. 6,350,123; U.S. Pat. No. 6,389,193; U.S. Pat. No. 6,544,256; U.S. Pat. No. 6,561,803; U.S. Pat. No. 6,567,582; U.S. Pat. No. 6,610,053; U.S. Pat. No. 6,616,447; U.S. Pat. No. 6,616,451; U.S. Pat. No. 6,669,685; and U.S. Pat. No. 6,744,790. For example, one implementation of a delivery system coupled to a laser base unit may be useful for optimizing or maximizing a cutting effect of a laser. The laser output can be directed, for example, into fluid (e.g., an air and water spray or an atomized distribution of fluid particles from a spray water connection 55 and/or spray air connection 50) above a target surface. An apparatus including a delivery system for directing laser energy into an atomized distribution of fluid particles above a target surface is disclosed in the above-referenced U.S. Pat. No. 5,574,247. Large amounts of laser energy can be imparted into the fluid (e.g., atomized fluid particles) which can comprises water, to thereby expand the fluid (e.g., fluid particles) and apply disruptive (e.g., mechanical) cutting forces to the target surface.

In view of the foregoing, it will be understood by those skilled in the art that the methods of the present invention can facilitate operation of electromagnetic energy devices, and in particular medical laser devices. The above-described embodiments have been provided by way of example, and the present invention is not limited to these examples. Other mechanisms of contact between connector and adapter 100 (FIG. 2) or apparatus 300 (FIG. 4) may be employed. For example, magnetic, inductive, radio frequency, or optical methods/devices may be used in some embodiments. The present invention can have applicability in the context of various configurations and components, such as connectors and adapters, for providing discrimination between various types of users, processes, protocols and/or equipment. Although described in the context of a multi-state system for providing discrimination between two states (e.g., general-purpose and specialized equipment), more than two states of various types may be provided in modified embodiments and/or applications (such as applications for providing user, device, process, or system identification). An example of such a modified application can comprise implementation of radio-frequency identification (RFID) implementations in which, for example, RF signals are provided in addition to or as an alternative to the above described pins and pin-contacting surfaces. For example, in embodiments wherein the above-discussed pins and pin-contacting surfaces are omitted, circuitry and/or microprocessors may be used for facilitating communication (e.g., RFID communication using any type of communication protocol, goal, or functionality known to those skilled in the art) between devices and/or users for various purposes including those set forth above and others, such as the discrimination between (e.g., identification of) different users and/or equipment. Multiple variations and modification to the disclosed embodiments will occur, to the extent not mutually exclusive, to those skilled in the art upon consideration of the foregoing description. Additionally, other combinations, omissions, substitutions and modifications will be apparent to the skilled artisan in view of the disclosure herein. Accordingly, the present invention is not intended to be limited by the disclosed embodiments, but is to be defined by reference to the appended claims.

What is claimed is:

1. An identification connector incorporated into a delivery system that connects to an electromagnetic energy housing, wherein the electromagnetic energy housing includes an active identification device capable of receiving information from the identification connector, an electromagnetic energy power coupling capable of providing electromagnetic energy power to the identification connector, and a plurality of ancillary couplings, the identification connector comprising:
a passive identification device capable of receiving operation power from, and providing indication information to, the active identification device;
an electromagnetic energy beam delivery guide connection capable of receiving electromagnetic energy power from the electromagnetic energy power coupling; and
a plurality of ancillary connections capable of connecting to the plurality of ancillary couplings.

2. The identification connector as set forth in claim 1, wherein:
the electromagnetic energy housing comprises a laser housing;
the electromagnetic energy power coupling comprises a laser power coupling; and
the electromagnetic energy beam delivery guide comprises a laser beam delivery guide.

3. The identification connector as set forth in claim 1, wherein the indication information comprises identification information.

4. The identification connector as set forth in claim 1, wherein the indication information comprises a calibration factor.

5. The identification connector as set forth in claim 1, wherein the indication information comprises a usage count.

6. The identification connector as set forth in claim 1, wherein the active identification device is capable of:
receiving identification information according to the passive identification device; and
receiving a calibration factor for electromagnetic energy power delivery according to the passive identification device.

7. The identification connector as set forth in claim 1, wherein the plurality of ancillary connections comprises:
a spray air connection;
a spray water connection;
a cooling air connection;
an illumination light connection;
an excitation light connection; and
a feedback channel connection.

8. The identification connector as set forth in claim 7, wherein:
the spray air connection is capable of receiving spray air from the electromagnetic energy housing;
the spray water connection is capable of receiving spray water from the electromagnetic energy housing;
the cooling air connection is capable of receiving cooling air from the electromagnetic energy housing;
the illumination light connection is capable of receiving light from the electromagnetic energy housing;
the excitation light connection is capable of receiving light from the electromagnetic energy housing; and
the feedback channel connection is capable of providing feedback to the electromagnetic energy housing.

9. An identification connector, comprising:
an electromagnetic energy beam delivery guide connection capable of receiving an electromagnetic energy bean from an electromagnetic energy housing; and
a plurality of ancillary connections, the electromagnetic energy beam delivery guide connection and the plurality of ancillary connections being integrated with a delivery system and being capable of connecting to an electromagnetic energy housing, the electromagnetic energy housing being capable of transmitting power into at least one of the plurality of ancillary connections; and
a passive identification device capable of receiving operation power from, and for sending an indication to, an electromagnetic energy housing.

10. The identification connector as set forth in claim 9, wherein:
the electromagnetic energy housing comprises a laser housing;
the electromagnetic energy power coupling comprises a laser power coupling; and
the electromagnetic energy beam delivery guide comprises a laser beam delivery guide.

11. The identification connector as set forth in claim 9, wherein the indication comprises identification information.

12. The identification connector as set forth in claim 9, wherein the indication comprises a calibration factor for electromagnetic energy power delivery.

13. The identification connector as set forth in claim 9, wherein the indication comprises a usage count.

14. The identification connector as set forth in claim 9, wherein the passive identification device is capable of providing information to an active identification device disposed in the electromagnetic energy housing.

15. The identification connector as set forth in claim 9, wherein the plurality of ancillary connections comprises:
a spray air connection;
a spray water connection;
a cooling air connection;
a illumination light connection;
an excitation light connection; and
a feedback channel connection.

16. The identification connector as set forth in claim 15, wherein:
the spray air connection is capable of receiving spray air from the electromagnetic energy housing;
the spray water connection is capable of receiving spray water from the electromagnetic energy housing;
the cooling air connection is capable of receiving cooling air from the electromagnetic energy housing;
the illumination light connection is capable of receiving illumination light from the electromagnetic energy housing;
the excitation light connection is capable of receiving excitation light from the electromagnetic energy housing; and
the feedback channel connection is capable of sending feedback to the electromagnetic energy housing.

17. The identification connector as set forth in claim 9, wherein the electromagnetic energy beam delivery guide is capable of receiving electromagnetic energy power from the electromagnetic energy housing.

18. A method of connecting an electromagnetic energy housing to a delivery system, the method comprising:
providing an identification connector comprising a first identification device, the identification connector being connected to and forming a portion of the delivery system;
connecting the identification connector to the electromagnetic energy housing;
providing operation power to the first identification device to thereby activate the first identification device; and
receiving an indication according to the first identification device.

19. The identification connector as set forth in claim 18, wherein:
the electromagnetic energy housing comprises a laser housing;
the electromagnetic energy power coupling comprises a laser power coupling; and
the electromagnetic energy beam delivery guide comprises a laser beam delivery guide.

20. The method as set forth in claim 18, further comprising:
providing in the identification connector an electromagnetic energy beam delivery guide connection; and
providing in the identification connector a plurality of ancillary connections.

21. The method as set forth in claim 20, wherein the providing of a plurality of ancillary connections comprises providing:
a spray air connection;
a spray water connection;
a cooling air connection;
a illumination light connection;
an excitation light connection; and
a feedback channel connection.

22. The method as set forth in claim 20, further comprising:
providing in the electromagnetic energy housing a second identification device capable of receiving the indication according to the first identification device;
providing in the electromagnetic energy housing a coupling capable of providing electromagnetic energy power to the electromagnetic energy beam delivery guide; and
providing in the electromagnetic energy housing a plurality of couplings capable of connecting to the plurality of ancillary connections.

23. The method as set forth in claim 22, wherein the providing of a plurality of couplings comprises:
providing a coupling of a source of spray air;
providing a coupling of a source of spray water;
providing a coupling of a source of cooling air;
providing a coupling of illumination light;
providing a coupling of excitation light; and
providing a feedback detector.

24. The method as set forth in claim 18, wherein the receiving of an indication comprises receiving identification information according to the first identification device.

25. The method as set forth in claim 24, further comprising:
verifying proper connection between the identification connector and the electromagnetic energy housing;
verifying that the delivery system is authorized; and
disabling delivery of electromagnetic energy power when the delivery system is not authorized.

26. The method as set forth in claim 18, wherein the receiving of an indication comprises receiving a calibration factor for electromagnetic energy power delivery.

27. The method as set forth in claim 26, further comprising adjusting at least one output parameter of the electromagnetic energy housing, based upon the calibration factor received by the first identification device.

28. The method as set forth in claim 18, wherein the receiving of an indication comprises receiving a usage count.

29. The method as set forth in claim 28, further comprising initiating preventive maintenance according to the usage count.
